(19) United States
(12) Patent Application Publication
(10) Pub. No.: US 2005/0176973 A1
(43) Pub. Date: Aug. 11, 2005

(54) Method for producing pure trioxane

(76) Inventors: Katrin Friese, Mannheim (DE); Matthias Rauls, Limburgerhof (DE); Reinhard Freyhof, Frankenthal (DE); Thorsten Friese, Mannheim (DE); Harald Armbruster, Limburgerhof (DE); Hartmut Zeiner, Ludwigshafen (DE); Gitta Egbers, Osnabruck (DE); Eckhard Strofer, Mannheim (DE); Ludwig E. Heck, Edingen-Neckarhausen (DE); Peter Hildenbrand, Karlsruhe (DE)

Correspondence Address: NOVAK DRUCE DELUCA & QUIGG, LLP, 1300 EYE STREET NW, SUITE 400 EAST, WASHINGTON, DC, 20005, US

(21) Appl. No.: 10/514,490

(22) PCT Filed: May 13, 2003
(86) PCT No.: PCT/EP03/04968

(30) Foreign Application Priority Data
May 17, 2002 (DE) 102 22 163

(51) Int. Cl. C07D 323/06

(52) U.S. Cl. 549/368

(57) ABSTRACT

A process for preparing trioxane by trimerization of formaldehyde in aqueous acidic solution and subsequently removing trioxane from a mixture substantially comprising trioxane, water and formaldehyde (crude trioxane product), which comprises a) distilling the crude trioxane product from the trimerization, b) crystallizing the trioxane out and c) removing it from the mother liquor and d) subsequently melting it and e) distilling off further by-products.

The invention relates to an improved process for preparing pure trioxane.

When trioxane is prepared (see, for example, DE-A 1543390), a mixture is obtained which essentially comprises trioxane, water and formaldehyde. Trioxane is extractively removed from this mixture with the aid of an azeotroping agent, for example a chlorinated azeotroping agent such as methylene chloride, or benzene. Further components of the mixture in subordinate quantities are generally formic acid, methylal and dimethoxydimethyl ether and also methanol and methyl formate. In a subsequent distillation, the azeotroping agent is recovered and fed back to the extraction. In this process (sees for example, EP-A 583 907), large amounts of azeotroping agent have to be used and recovered at high energy cost. Emissions which inevitably occur have to be disposed of in a costly and inconvenient manner, since methylene chloride and benzene are classified as dangerous pollutants.

The extract is neutralized; the resulting salts are washed out, since they influence the polymerization of trioxane even in small concentrations. In a subsequent purification of the extract by distillation, the extractant and interfering secondary components are removed in order to provide polymerizable trioxane. Further distillation steps are necessary for the workup of the aqueous formaldehydic raffinate, of the extractant and also of the salt-containing waste liquor.

In this process, the extraction is a particularly costly and inconvenient step in purifying trioxane and is additionally associated with the use of chlorine compounds, for example methylene chloride, which are undesired from an ecological point of view. These are associated with strict environmental conditions. Depending on the extractant, sometimes considerable amounts of trioxane remain in the aqueous phase in the extraction. This results in large circulation streams. The organic solvent introduces a further material into the process. This results in a further workup of the organic phase being required which is associated with losses of trioxane. The washing steps included in the extraction stage alone furthermore require 25% of the total amount of demineralized water used in the process, so that omission of the extraction would also allow the waste water streams to be worked up to be reduced.

DE-A 350 86 68 (Hoechst A G., Frankfurt) discloses the preparation of highly pure trioxane from an aqueous formaldehyde/trioxane mixture by multistage crystallization which is at least partially carried out as a melt crystallization (layer crystallization). This is carried out using either directly the aqueous mixture resulting from a trioxane synthesis or an already prepurified, substantially water-free mixture. According to the teaching of the document, useful trioxane yields are only obtained at trioxane concentrations in the starting mixture of at least 50% by weight, preferably at least 95% by weight, owing to the location of the eutectic point. When the water content of the starting mixture is high (from 30 to 50% by weight), the first stage of the crystallization is carried out as a solution crystallization. In order to generate a trioxane concentration in the starting mixture of at least 50% by weight, further workup steps are necessary after the synthesis. Layer crystallization processes are frequently used when the starting concentrations of the product of value are relatively high (>95% by weight). In order to attain a trioxane concentration of 95% by weight in the starting mixture, the trioxane/water azeotrope has to be passed over. This passing over of the azeotrope results in an important objective of the crystallization, i.e. the saving of process steps by direct crystallization after the synthesis step, not being achieved. In addition, further subsequent treatments of the trioxane are necessary even when the purity is higher in order to attain the necessary low water contents (<50 ppm) for the subsequent polymerization.

The separation of trioxane from gaseous mixtures comprising formaldehyde by absorption in alcoholic liquids and subsequent crystallization is described, for example, in DE-A 19833620 and EP-A 976743. Gaseous formaldehyde and trioxane are preferably dissolved in a mono- or polyhydric alcohol. Trioxane is crystallized out of the solution either in a layer crystallization or in a continuous suspension process and then removed.

In a layer crystallization process, crystals are frozen out on cooled surfaces in the form of coherent, strongly adhering layers. The solid/liquid separation is effected by simply allowing the residual melt to run off; the purified crystals are melted. High purities and yields are generally only obtained by repeated, cyclical crystallization. In every stage, the heat of crystallization in the freezing out has to be removed and then reintroduced in the subsequent melting. In addition, the apparatus itself also has to be heated and cooled again in each case. The trioxane yield in the abovementioned processes is only about 51.4%.

An alternative to layer crystallization described in EP-A 976 743 is a process for suspension crystallization of trioxane.

In the crystallization process described in the prior art, gaseous trioxane is processed. The crystallization is necessarily preceded by the absorption of trioxane in a suitable solution. However, as already illustrated, additional process steps and solvents are uneconomical for the process and therefore disadvantageous in the process described.

EP-A 573 850 discloses the preparation of polymerizable trioxane of high purity by multistage crystallization with additives. The addition of additives (alkaline organic compounds, for example tertiary amines) effectively prevents the formation of “flocks” which, after several operating hours and crystallization cycles at high trioxane contents (>95% by weight), are suspected to result from paraformaldehyde formation. The additives are removed in sweating and/or washing to such an extent that they are no longer analytically detectable and do not impair the polymerization of the pure trioxane. The mixture used for the crystallization in this case already has a trioxane content of 94% by weight. A two-stage layer crystallization delivers trioxane having a purity of 99.9% by weight. The layer crystallization described has the abovementioned disadvantages of high energy demands and the necessity of a high starting concentration of trioxane.

According to EP-A 248 487, polyoxymethylene may be prepared from trioxane which has been concentrated by rectification after the synthesis and then removed from the aqueous synthesis mixture by extraction using aliphatic hydrocarbons. The extract (trioxane in organic extraction solvent) is subsequently crystallized. The trioxane purified by crystallization is resuspended and polymerized from the suspension. The disadvantages of extractive removal have already been described above.

DE 19 842 579 describes a process for removing trioxane from liquid mixtures by highly selectively transferring the trioxane into the gas phase by evaporation and subsequently recovering it in solid or liquid form by cooling and condensation or desublimation. A problem with such an evaporation of trioxane at atmospheric pressure is the high thermal stress. It is therefore sensible although uneconomical to use a carrier gas with which the trioxane is stripped out of the liquid mixture. The process described thus has no recognizable advantages for the workup of an aqueous trioxane solution, particularly because the trioxane concentration is beyond the azeotrope of trioxane and water even before the two purification steps of evaporation and desublimation, and additional further process steps are required to generate pure trioxane.

U.S. Pat. No. 2,465,489 discloses a process for recovering trioxane from aqueous solutions by crystallization. Residues of formaldehyde are removed by washing with methanol/ethanol. However, this results in about 30% of the trioxane likewise going into solution which correspondingly reduces the trioxane yield. A further disadvantage is the high starting concentration of at least about 50% by weight of trioxane in the mixture which may only be attained by additional preceding process steps.

It is an object of the present invention to provide a process for preparing and subsequently removing trioxane which allows very substantial recovery of the trioxane and requires very little additional solvent and few process steps and also produces fewer by-products. The trioxane obtained shall be highly pure and obtainable in high yields, and the water content in particular shall be very low (below 50 ppm), since there is otherwise no polymerizability.

We have found that this object is achieved by a process for preparing trioxane by trimerizing of formaldehyde in aqueous acidic solution and subsequently removing trioxane from a mixture substantially comprising trioxane, water and formaldehyde (crude trioxane product), which comprises
a) distilling the crude trioxane product from the trimerization,
b) crystallizing the trioxane out and
c) removing it from the mother liquor and
d) subsequently melting it and
e) distilling off further by-products.

The preparation of trioxane from formaldehyde (trimerization) in the presence of aqueous acidic solutions is known to those skilled in the art, for which reason further information on this subject is superfluous. The crude trioxane product (mixture after the trimerization) comprises, in accordance with the invention, trioxane contents of from 30 to 55% by weight, preferably from 35 to 46% by weight at a formaldehyde level of from 15 to 30% by weight, preferably from 18 to 25% by weight, and a water level of from 25 to 40% by weight, preferably from 28 to 38% by weight.

Trimerization by-products in the crude trioxane product are generally methanol (<5% by weight), methylformate (<5% by weight), methylal (<4% by weight), formic acid (<3% by weight), tetroxane (<1% by weight) and dimethoxydimethyl ether (=DOE, <1% by weight) and also high-boiling secondary components.

In the removal procedure according to the invention, the crude trioxane product from the trimerization is initially distilled in a stage a) and the low-boilers such as methyl formate, methylal, methanol, DOE and also small amounts of water and formaldehyde are removed.

The distillation may take place in one or more columns. The top temperature is generally >40° C., preferably >60° C. Preference is given to carrying out the distillation at atmospheric pressure or at a slightly elevated pressure of up to 2.5 bar.

After stage a), the bottom product comprises predominantly from 30 to 55% by weight, preferably from 36 to 47% by weight, of trioxane, from 15 to 30% by weight, preferably from 18 to 25% by weight, of formaldehyde, and from 25 to 40% by weight, preferably from 28 to 38% by weight, of water, and also small fractions of the abovementioned by-products.

This mixture (bottom product) is discharged from the column (or columns) according to the invention and transferred to suitable apparatus for the crystallization stage b). Examples of useful apparatus include cooling disk crystallizers, tubular crystallizers and scraped-surface coolers.

The crystallization b) may be carried out either batchwise or else continuously or semicontinuously, and in one or more stages. A preferred procedure is continuous crystallization which is in particular carried out in one stage. Stage b) may be a melt and/or solution crystallization.

A particularly preferred embodiment of the crystallization stage b) in the removal process according to the invention is suspension crystallization.

The temperatures of the crystallization stage b) of the process according to the invention are from −10 to +65° C., preferably from 0 to 40° C. Preferred maximum cooling rates in the batchwise method are 15 K/hour, in particular 10 K/hour. In the continuous method, preference is given to implementing a temperature gradient along the crystallizer. In the methods, preference is given in particular to end temperatures of s 20° C., preferably ≦15° C. In general, stage b) is advantageously carried out at atmospheric pressure, but operation may also be effected under elevated pressures of up to 2.5 bar or reduced pressures of down to 0.3 bar, although no process engineering advantages are to be expected.

After stage b) of the process according to the invention, the mother liquor is removed from the crystals (stage c) and advantageously worked up distillatively to recover formaldehyde and trioxane. The predominantly formaldehydic stream from the distillation of the mother liquor is recycled into the trimerization, and the predominantly trioxanic stream into the low boiler removal a).

Useful apparatus for the crystal removal includes any type of centrifuge, for example skimmer centrifuges and particularly pusher centrifuges, belt filters or, more preferably, washing columns. These are advantageous because stage c) and also the following stage d) may be carried out in one apparatus and the crystals may optionally also-be washed by means of countercurrent washing of the crystals.

After the crystallization, the mother liquor generally comprises a maximum of up to 20% by weight of trioxane, from 30 to 40% by weight of formaldehyde, and from 35 to 55% by weight of water and also small proportions of methanol, formic acid, methyl formate, methylal, butanediol formal and tetroxane.

After the crystallization b), the removed crystals already comprise >92% by weight of trioxane, from 1 to 5% by weight of formaldehyde, from 1 to 7% by weight of water and also <0.3% by weight of high boilers, for example tetroxane, TOE=trimethoxydimethyl ether.

The crystals may optionally be washed after the removal c). Useful washing media are water and/or alcohols such as methanol, ethanol, 1- or 2-propanol. Preference is given to carrying out the washing of the crystals at washing medium temperatures of ≦20° C., preferably ≦5° C. The volumetric ratio of crystals to washing liquid is generally from 1:0.5 to 1:3, preferably 1:1.

Washing with water causes the proportion of water to increase slightly, while the formaldehyde proportion generally falls below 0.5% by weight. The trioxane content remains very substantially constant at >92% by weight.

In a particularly preferred procedure of washing of the crystals, washing is effected first with water and then with alcohols, preferably methanol.

The crystals obtained in this manner comprise at least 98% by weight of trioxane, less than 0.1% by weight of formaldehyde and also ≦0.5% by weight of water.

The crystals are melted in the subsequent stage d) of the process according to the invention.

Useful apparatus for stage d) includes insulated tanks with melting circuits (heat exchangers using steam or condensate), the liquid phase circuit of a washing column and sublimers.

According to the invention, the melted crystals are introduced into one or more columns and redistilled (stage e).

In stage e), TOE, DOE, tetroxane and water in particular are removed.

The distillation stage e) is operated at a pressure of from 0.3 to 2 bar, preferably at atmospheric pressure. The temperature of the liquid feed at atmospheric pressure is from 62 to 120° C. However, it is also possible to operate the column using an entirely or partially gaseous feed (feed temperature a 120° C. at atmospheric pressure).

In a low pressure column, the pressure is generally up to 0.3 bar. At temperatures of from 62 to 80° C., the feed is liquid, and at temperatures of >80° C. at least partially gaseous.

In the process according to the invention, the aqueous phase (top product) from the purifying distillation (stage e) may be recycled upstream of stage a), and the high boilers from stage e) may advantageously be recycled into the trimerization.

After the purifying distillation e), the trioxane is discharged from stage e) and is transferred to the polymerization process.. In the process according to the invention, no additional components are required. The removal is effected in few steps and very substantially without the formation of further by-products. Furthermore, a product of high purity is obtained which, according to the method of the invention, consists of at least 99.5% by weight of trioxane. The proportion of water, which is decisive for the polymerization, is <50 ppm, the formic acid content <5 ppm, the DOE content <100 ppm, and the TOE content from 100 to 200 ppm.

### EXAMPLES

### Example 1

Trimerization of Formaldehyde, Low Boiler Removal, Batchwise Crystallization of Trioxane Without Washing Step (End Temperature: 21° C.), Purifying Distillation

Aqueous formaldehyde solution (49% by weight of formaldehyde) was concentrated in a falling-film evaporator at 68° C. and 0.25 bar. The concentrated formaldehyde solution (63% by weight of formaldehyde, 34% by weight of water, remainder: mainly methanol) was fed into the bottom of the synthesis column at atmospheric pressure and a top temperature of 98° C. The catalyst used was sulfuric acid. Trioxane was concentrated toward the top of the column to about 35% by weight. In a downstream distillation column, low boilers were removed. The low boiler stream removed consisted of 56% by weight of methyl formate, 26% by weight of methylal, 9% by weight of methanol, 6% by weight of DOE, and also small amounts of water and formaldehyde. The resulting mixture of trioxane, formaldehyde and water (35.5% by weight of trioxane, 26.7% by weight of formaldehyde, 36% by weight of water, remainder: secondary components, mainly methanol) was crystallized batchwise in a tubular crystallizer equipped with a helical stirrer (laboratory version of a cooling disk crystallizer, capacity: 5 1) starting at 38° C. (cooling rate: 3 K/h, end temperature: 21° C.). The crystals were removed from the mother liquor on a screen bowl centrifuge at 2000 rpm within 3 min. The unwashed crystals had the following composition: 97.3% by weight of trioxane, 1.2% by weight of formaldehyde, 1.4% by weight of water, 0.1% by weight of high boilers. The crystallized trioxane was melted at atmospheric pressure in an isolated tank under a nitrogen atmosphere and transferred at a temperature of 95° C. to a distillation column which was operated at atmospheric pressure. Trioxane was recovered as a vaporous sidestream and then condensed. The purity of the trioxane after distillation was >99.5% by weight and there was also <50 ppm of water, <5 ppm of formic-acid, <100 ppm of DOE and 160 ppm of TOE.

### Example 2

Batchwise Crystallization, Washing with Water (end Temperature: 10° C.)

A mixture of trioxane, formaldehyde and water (35.5% by weight of trioxane, 26.7% by weight of formaldehyde, 36% by weight of water) was crystallized in a similar manner to the procedure in Example 1. The crystallization end temperature was 10° C. The crystals were washed with water in a ratio of 1:1 (volumetric) and, after washing, had a purity of 95.3% by weight (0.2% by weight of formaldehyde, 4.4% by weight of water, 0.1% by weight of high boilers). The yield of trioxane was 73%.

### Example 3

Continuous Crystallization at 10° C., Without Washing Step

A mixture of trioxane, formaldehyde and water (36.7% by weight of trioxane, 26.4% by weight of formaldehyde, 35.4% by weight of water) was continuously crystallized in a tubular crystallizer equipped with a helical stirrer at 10° C. with a residence time of 1 h. The crystals were removed from the mother liquor on a screen bowl centrifuge at 2000 rpm within 30 s. The unwashed crystals had the following composition: 94.5% by weight of trioxane, 2.7% by weight of formaldehyde, 5% by weight of water.

### Example 4

Continuous Crystallization at 10° C., Washing with Water

A mixture of trioxane, formaldehyde and water (36.7% by weight of trioxane, 26.4% by weight of formaldehyde, 35.4% by weight of water) was crystallized in a similar manner to Example 3, except that a residence time of 2 h was employed. After the mother liquor was removed, the crystals were washed with water in a ratio (volumetric) of 1:1 (washing step: 30 s, centrifugal drying: 180 s, each at 2000 rpm). The washed crystals have a purity of 92.9% by weight (0.1% by weight of formaldehyde, 7% by weight of water). The formaldehyde content in the crystals was markedly reduced by the expulsive washing with water. The higher water content was attributed to adhering washing water. The trioxane yield was 79%.

### Example 5

Continuous Crystallization at 10° C., Washing with Water and Methanol

A mixture of trioxane, formaldehyde and water (36.7% by weight of trioxane, 26.4% by weight of formaldehyde, 35.4% by weight of water) was crystallized in a similar manner to Example 4. After the removal of the mother liquor, the crystals were washed twice with water and once with methanol in a ratio of 1:1 (volumetric) (each washing step: 30 s, centrifugal drying: 180 s, each at 2000 rpm). The crystals were then virtually formaldehyde- and water-free and had the following composition: 99% by weight of trioxane, 0.02% by weight of formaldehyde, 0.3% by weight of water. Owing to the more extensive dissolving of crystals on repeated washing, the trioxane yield fell to 53%.

1. A process for preparing trioxane by trimerizing formaldehyde in aqueous acidic solution and subsequently removing trioxane from a mixture substantially comprising trioxane, water and formaldehyde (crude trioxane product), which comprises
a) distilling the crude trioxane product from the trimerization,
b) crystallizing the trioxane out and
c) removing it from the mother liquor and
d) subsequently melting it and
e) distilling off further by-products.

2. A process as claimed in claim 1, wherein the trioxane is discharged from stage e) and is transferred to a polymerization stage.

3. A process as claimed in claim 1, wherein the mother liquor of stage c) is distilled to recover formaldehyde and trioxane.

4. A process as claimed in claim 3, wherein the predominantly formaldehydic stream from the distillation of the mother liquor is recycled into the trimerization and the predominantly trioxanic stream is recycled into stage a).

5. A process as claimed in claim 1 to 4, wherein stage b) is carried out as a suspension crystallization.

6. A process as claimed in claim 1, wherein stage b) of the process is carried out in one stage.

7. A process as claimed in claim 1, wherein an end temperature of <20° C. is set in stage b) of the process.

8. A process as claimed in claim 1, wherein the crystallized trioxane after the removal of the mother liquor (stage c) is washed and subsequently melted (stage d).

9. A process as claimed in claim 1, wherein the crystallized trioxane is washed with water and/or alcohols.

10. A process as claimed in claim 7, wherein the crystallized trioxane is washed at temperatures of ≦20° C.
